(19) United States
(12) Patent Application Publication
(10) Pub. No.: US 2025/0338815 A1
(43) Pub. Date: Nov. 6, 2025

(54) MILKING SYSTEM

(71) Applicant: S.C.R. (ENGINEERS) LIMITED, Netanya (IL)

(72) Inventors: Mordehay COHEN, Netanya (IL); Yoni KAMINSKY, Netanya (IL); Yosef ARTZI, Netanya (IL); Taras KHOMITSKY, Netanya (IL); Eden WEINBERG, Netanya (IL); Alexander GERSHIKOV, Netanya (IL)

(21) Appl. No.: 19/269,072

(22) Filed: Jul. 15, 2025

Related U.S. Application Data
(63) Continuation of application No. 17/735,123, filed on May 3, 2022, now Pat. No. 12,402,596.

(51) Int. Cl. A01J 7/02; A01J 5/01; B08B 9/032; G01F 1/661; G01N 33/04

(52) U.S. Cl. CPC A01J 7/02; A01J 5/01; B08B 9/0325; G01F 1/661; G01N 33/04

(57) ABSTRACT

A wash fluid conservation system and method are disclosed. Temperature values are obtained for one or more given milkmeters in a milkmeter system for one or more sampling periods during a given iteration of a washing process for cleaning the milkmeter system. The temperature values are indicative of a temperature of the wash fluid that is flowing through the respective given milkmeter. If the temperature values for a respective sampling period are greater than or equal to a predefined temperature prior to a designated time for completion of the given iteration, one or more actions are performed to reduce the wash fluid that is used in at least one of the given iteration or a subsequent iteration of the washing process to be less than an amount of the wash fluid designated to be used in the washing process in advance of the given iteration.

### TECHNICAL FIELD

The invention relates to milkmeter systems, including milkmeters with milk channels and feed inlets coupled thereto, and systems and methods for conserving wash fluid in washing processes for cleaning milkmeter systems.

### BACKGROUND

Milkmeters in a milkmeter system measure a quantity of milk that is extracted from non-human mammals (e.g., cows, sheep, goats, etc.) over the course of a milking session. Food safety and public health regulations require that a milkmeter system be cleaned on a regular basis, for example, to avoid an accumulation of milk soil and/or bacteria in the milkmeter system. In order to clean the milkmeter system, water or wash fluid must be heated and then streamed throughout the milkmeter system. An object of the present disclosure is to ensure that the milkmeter system is cleaned while conserving resources, such as water, wash fluid, electricity, and/or power.

References considered to be relevant as background to the presently disclosed subject matter are listed below. Acknowledgement of the references herein is not to be inferred as meaning that these are in any way relevant to the patentability of the presently disclosed subject matter.

U.S. Patent Application Publication No. 2021/0262869 (“Pinsky et al.”), published on Aug. 26, 2021, discloses a monitoring milk meter, which is able to monitor the livestock being milked, as well as general procedures performed in the milking farm, such as a Cleaning in Place (CIP) procedure.

### GENERAL DESCRIPTION

In accordance with a first aspect of the presently disclosed subject matter, there is provided a device comprising: a milk channel having a first part and a second part, wherein a first value of at least one optical property of the first part is different than a second value of the optical property of the second part.

In some cases, at least a section of the milk channel includes four sides, wherein a first side of the sides is substantially opposite a second side of the sides, wherein both the first side and the second side are of a rectangular cross-section, and wherein the second part of the milk channel comprises a first window, being at least a portion of the first side of the sides, and a second window, being at least a second portion of the second side of the sides.

In some cases, the milk channel is a straight milk channel.

In accordance with a second aspect of the presently disclosed subject matter, there is provided a feed inlet, configured to be coupled to a milk conducting hose and to a milk channel of a milkmeter, the feed inlet comprising: a curved pipe having a first end and a second end, the first end having a first opening, enabling a fluid that flows through a milk conducting hose to enter the curved pipe, and the second end having a second opening for enabling the fluid to exit the curved pipe and enter the milk channel, wherein an angle of curvature between the first end and the second end is less than about 90 degrees.

In some cases, the feed inlet is configured to be coupled to the milk conducting hose via a hollow adaptor that extends between the milk conducting hose and the feed inlet.

In some cases, the feed inlet is configured to be coupled to the milk channel by connecting the second end of the curved pipe to an upper end of the milk channel.

In some cases, the angle of curvature is selected to increase an amount of time in which the milk channel is filled with the fluid.

In some cases, the angle of curvature is about 60 degrees.

In accordance with a third aspect of the presently disclosed subject matter, there is provided a wash fluid conservation system comprising a processing circuitry configured to: for one or more given milkmeters of one or more milkmeters in a milkmeter system, obtain, for one or more sampling periods during a given iteration of a given washing process for cleaning the milkmeter system, temperature values that are indicative of a temperature of a wash fluid that is flowing through the respective given milkmeter; and in response to the temperature values for a respective sampling period of the sampling periods being greater than or equal to a predefined temperature prior to a designated time for completion of the given iteration, perform one or more actions to reduce an amount of the wash fluid that is used in at least one of the given iteration or a subsequent iteration of the given washing process, subsequent to the given iteration, to be less than a designated amount of the wash fluid designated to be used in the given washing process in advance of the given iteration.

In some cases, the temperature values are obtained from one or more temperature sensors that are included in the given milkmeters.

In some cases, the actions include stopping the given iteration before the designated time.

In some cases, the given iteration is immediately stopped upon the temperature values for the respective sampling period being greater than or equal to the predefined temperature.

In some cases, the actions include designating a reduced amount of the wash fluid to be used in the subsequent iteration in advance of the subsequent iteration, the reduced amount being less than the designated amount.

In some cases, the processing circuitry is further configured to: calculate or determine an accumulated amount of the wash fluid that has flowed through the milkmeter system either: (a) during the given iteration or (b) until the end of the respective sampling period; wherein the reduced amount is designated to be greater than or equal to the accumulated amount.

In accordance with a fourth aspect of the presently disclosed subject matter, there is provided a wash fluid conservation method, comprising: for one or more given milkmeters of one or more milkmeters in a milkmeter system, obtaining, for one or more sampling periods during a given iteration of a given washing process for cleaning the milkmeter system, temperature values that are indicative of a temperature of a wash fluid that is flowing through the respective given milkmeter; and in response to the temperature values for a respective sampling period of the sampling periods being greater than or equal to a predefined temperature prior to a designated time for completion of the given iteration, performing one or more actions to reduce an amount of the wash fluid that is used in at least one of the given iteration or a subsequent iteration of the given washing process, subsequent to the given iteration, to be less than a designated amount of the wash fluid designated to be used in the given washing process in advance of the given iteration.

In some cases, the temperature values are obtained from one or more temperature sensors that are included in the given milkmeters.

In some cases, the actions include stopping the given iteration before the designated time.

In some cases, the given iteration is immediately stopped upon the temperature values for the respective sampling period being greater than or equal to the predefined temperature.

In some cases, the actions include designating a reduced amount of the wash fluid to be used in the subsequent iteration in advance of the subsequent iteration, the reduced amount being less than the designated amount.

In some cases, the method further comprises: calculating or determining an accumulated amount of the wash fluid that has flowed through the milkmeter system either: (a) during the given iteration or (b) until the end of the respective sampling period; wherein the reduced amount is designated to be greater than or equal to the accumulated amount.

### BRIEF DESCRIPTION OF THE DRAWINGS

In order to understand the presently disclosed subject matter and to see how it may be carried out in practice, the subject matter will now be described, by way of non-limiting examples only, with reference to the accompanying drawings. The dimensions of components and features shown in the drawings are chosen for convenience and clarity of presentation and are not necessarily to scale. In the drawings:

FIG. 1 is a schematic illustration of one example of a milk channel, in accordance with the presently disclosed subject matter;

FIG. 2 is a block diagram schematically illustrating one example of a wash fluid conservation system, in accordance with the presently disclosed subject matter;

FIG. 3 is a flowchart illustrating one example of a sequence of operations for reducing an amount of a wash fluid that is used in at least one iteration of a given washing process for cleaning a milkmeter system comprising one or more milkmeters, in accordance with the presently disclosed subject matter;

FIG. 4 is a schematic illustration of one example of a feed inlet configured to be coupled to a milk channel of a milkmeter, in accordance with the presently disclosed subject matter; and

FIG. 5 is a schematic illustration of one example of a coupling of a feed inlet to a milk conducting hose and to a milk channel of a milkmeter, in accordance with the presently disclosed subject matter.

### DETAILED DESCRIPTION

In the following detailed description, numerous specific details are set forth in order to provide a thorough understanding of the presently disclosed subject matter. However, it will be understood by those skilled in the art that the presently disclosed subject matter may be practiced without these specific details. In other instances, well-known methods, procedures, and components have not been described in detail so as not to obscure the presently disclosed subject matter.

In the drawings and descriptions set forth, identical reference numerals indicate those components that are common to different embodiments or configurations.

Unless specifically stated otherwise, as apparent from the following discussions, it is appreciated that throughout the specification discussions utilizing terms such as “calculating”, “obtaining”, “performing”, “stopping”, “designating” or the like, include actions and/or processes, including, inter alia, actions and/or processes of a computer, that manipulate and/or transform data into other data, said data represented as physical quantities, e.g. such as electronic quantities, and/or said data representing the physical objects. The terms “computer”, “processor” and “processing circuitry” should be expansively construed to cover any kind of electronic device with data processing capabilities, including, by way of non-limiting example, a personal desktop/laptop computer, a server, a computing system, a communication device, a smartphone, a tablet computer, a smart television, a processor (e.g. digital signal processor (DSP), a microcontroller, a field-programmable gate array (FPGA), an application specific integrated circuit (ASIC), etc.), a group of multiple physical machines sharing performance of various tasks, virtual servers co-residing on a single physical machine, any other electronic computing device, and/or any combination thereof.

As used herein, the phrase “for example,” “such as”, “for instance” and variants thereof describe non-limiting embodiments of the presently disclosed subject matter. Reference in the specification to “one case”, “some cases”, “other cases” or variants thereof means that a particular feature, structure or characteristic described in connection with the embodiment(s) is included in at least one embodiment of the presently disclosed subject matter. Thus the appearance of the phrase “one case”, “some cases”, “other cases” or variants thereof does not necessarily refer to the same embodiment(s).

It is appreciated that, unless specifically stated otherwise, certain features of the presently disclosed subject matter, which are, for clarity, described in the context of separate embodiments, may also be provided in combination in a single embodiment. Conversely, various features of the presently disclosed subject matter, which are, for brevity, described in the context of a single embodiment, may also be provided separately or in any suitable sub-combination.

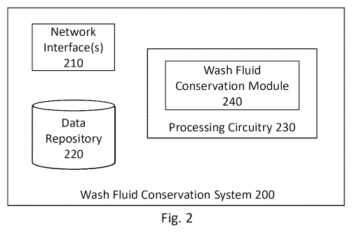

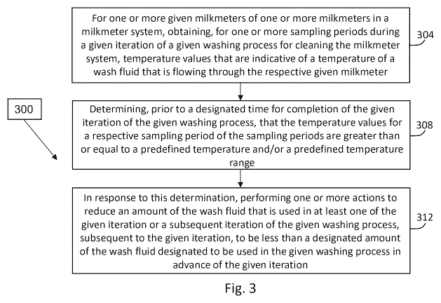

In embodiments of the presently disclosed subject matter, fewer, more and/or different stages than those shown in FIG. 3. In embodiments of the presently disclosed subject matter, one or more stages illustrated in FIG. 3 may be executed in a different order and/or one or more groups of stages may be executed simultaneously. FIG. 2 illustrates a general schematic of a system architecture, in accordance with an embodiment of the presently disclosed subject matter. Each module in FIG. 2 can be made up of any combination of software, hardware and/or firmware that performs the functions as defined and explained herein. The modules in FIG. 2 may be centralized in one location or dispersed over more than one location. In other embodiments of the presently disclosed subject matter, the system may comprise fewer, more, and/or different modules than those shown in FIG. 2.

Any reference in the specification to a method should be applied mutatis mutandis to a system capable of executing the method and should be applied mutatis mutandis to a non-transitory computer readable medium that stores instructions that once executed by a computer result in the execution of the method.

Any reference in the specification to a system should be applied mutatis mutandis to a method that may be executed by the system and should be applied mutatis mutandis to a non-transitory computer readable medium that stores instructions that may be executed by the system.

Any reference in the specification to a non-transitory computer readable medium should be applied mutatis mutandis to a system capable of executing the instructions stored in the non-transitory computer readable medium and should be applied mutatis mutandis to method that may be executed by a computer that reads the instructions stored in the non-transitory computer readable medium.

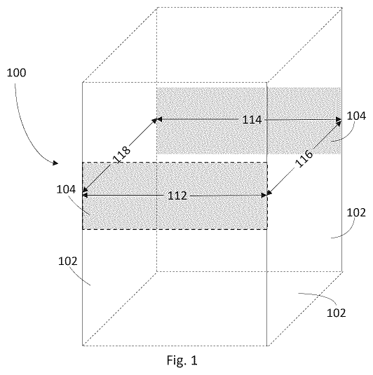

Attention is now drawn to FIG. 1, a schematic illustration of one example of a milk channel 100, in accordance with the presently disclosed subject matter.

In accordance with the presently disclosed subject matter, milk channel 100 is configured to enable a fluid (i.e, milk, a wash fluid, etc.) to flow therethrough. Milk channel 100 includes a first part 102 and a second part 104 (the second part 104 being shaded in grey in FIG. 1) wherein a first value of at least one optical property of the first part 102 is different than a second value of the optical property of the second part 104. In some cases, the milk channel 100 is a straight milk channel, as illustrated in FIG. 1. That is, the milk channel 100 is configured to enable a fluid (i.e., milk, a wash fluid, etc.) to flow through the milk channel 100 from a top of the milk channel 100 to a bottom of the milk channel 100 that is opposite, or substantially opposite, the top of the milk channel 100.

In some cases, at least a section of the milk channel 100 includes four sides 112, 114, 116 and 118; the second part 104 of the milk channel 100, shaded in grey; and at least some of the first part 102 of the milk channel 100. In some cases, all of the milk channel 100 to the exclusion of the second part 104 is the first part 102. In some cases, a first side 112 of the sides (112, 114, 116, 118) is substantially opposite a second side 114 of the sides (112, 114, 116, 118), and a third side 116 of the sides (112, 114, 116, 118) is substantially opposite a fourth side 118 of the sides (112, 114, 116, 118). In some cases, the first side 112 and the second side 114 are of a rectangular cross-section. In some cases, as illustrated in FIG. 1, the section of the milk channel 100 has a cubical structure. In some cases, the section of the milk channel 100 can have a structure other than a cubical structure, e.g., a tubular structure.

In some cases, the second part 104 of the milk channel 100 comprises a first window (shaded in grey in FIG. 1) being at least a portion of the first side 112 of the sides (112, 114, 116, 118), and a second window (shaded in grey in FIG. 1) being at least a portion of the second side 114 of the sides (112, 114, 116, 118). In some cases, the second part 104 of the milk channel 100 consists of the first window and the second window.

Attention is now drawn to FIG. 2, a block diagram schematically illustrating one example of a wash fluid conservation system 200, in accordance with the presently disclosed subject matter.

In accordance with the presently disclosed subject matter, wash fluid conservation system 200 can be configured to reduce an amount of wash fluid or water that is used in at least one iteration of a given washing process for cleaning a milkmeter system (not shown) comprising one or more milkmeters (not shown), the given washing process being at least part of a cleaning procedure for cleaning the milkmeter system. It is to be noted that all references in the present disclosure to the use of a wash fluid in a given washing process apply equally to the use of water in the given washing process. Moreover, it is to be noted that all references to a milkmeter in the present disclosure also apply to a milk sensor (i.e., a low-end milkmeter).

In some cases, wash fluid conservation system 200 can be configured to include one or more network interfaces 210. The network interfaces 210 are configured to connect the wash fluid conservation system 200 to one or more communications networks, thereby enabling the wash fluid conservation system 200 to send and receive data via the communications networks. In some cases, the communications networks can include an organizational (e.g., local) communications network for communications between milkmeters in the milkmeter system and the wash fluid conservation system 200. Additionally, or alternatively, in some cases, the communications networks can be configured to include an external communications network, such as the Internet, to enable the wash fluid conservation system 200 to communicate with other computerized devices that are external to the milkmeter system.

Wash fluid conservation system 200 can further comprise or be otherwise associated with a data repository 220 (e.g. a database, a storage system, a memory including Read Only Memory—ROM, Random Access Memory—RAM, and/or any other type of memory, etc.) configured to store data, including, inter alia, data received via the communications networks. In some cases, data repository 220 can be further configured to enable retrieval and/or update and/or deletion of the stored data. It is to be noted that in some cases, data repository 220 can be distributed.

Wash fluid conservation system 200 further comprises a processing circuitry 230. Processing circuitry 230 can be one or more processing units (e.g. central processing units), microprocessors, microcontrollers (e.g. microcontroller units (MCUs)) or any other computing devices or modules, including multiple and/or parallel and/or distributed processing units, which are adapted to independently or cooperatively process data for controlling relevant resources of the wash fluid conservation system 200 and for enabling operations related to resources of the wash fluid conservation system 200.

Processing circuitry 230 can be configured to include a wash fluid conservation module 240 for reducing an amount of the wash fluid that is used in at least one iteration of a given washing process for cleaning the milkmeter system, as detailed further herein, inter alia with reference to FIG. 3.

Attention is now drawn to FIG. 3, a flowchart illustrating one example of a sequence of operations 300 for reducing an amount of a wash fluid that is used in at least one iteration of a given washing process for cleaning a milkmeter system (not shown) comprising one or more milkmeters (not shown), in accordance with the presently disclosed subject matter.

In accordance with the presently disclosed subject matter, wash fluid conservation system 200 can be configured, e.g. using wash fluid conservation module 240, to obtain, for one or more given milkmeters of the milkmeters in the milkmeter system, temperature values that are indicative of a temperature of a wash fluid that is flowing through the respective given milkmeter during a given iteration of a given washing process of one or more washing processes for cleaning the milkmeter system. The temperature values are obtained for one or more sampling periods during the given iteration (block 304). In some cases, the sampling periods can be sampling instances.

The temperature values can be obtained from one or more temperature sensors (not shown). In some cases, the temperature sensors can be included in the given milkmeters. In some cases, the temperature sensor associated with a respective milkmeter of the milkmeters in the milkmeter system can be used to provide temperature values during a milking session for milking a non-human mammal, the temperature values being indicative of a temperature of milk that flows through the respective milkmeter during the milking session.

In some cases, one or more of the temperature sensors can be thermopiles. In some cases, all of the temperature sensors can be thermopiles.

Wash fluid conservation system 200 can be further configured, e.g. using wash fluid conservation module 240, to determine, prior to a designated time for completion of the given iteration of the given washing process, that the temperature values for a respective sampling period of the sampling periods are greater than or equal to a predefined temperature, and/or a predefined temperature range (block 308).

In response to this determination, wash fluid conservation system 200 can be configured, e.g. using wash fluid conservation module 240, to perform one or more actions to reduce an amount of the wash fluid that is used in at least one of the given iteration or a subsequent iteration of the given washing process, subsequent to the given iteration. The amount of the wash fluid is reduced to be less than a designated amount of the wash fluid designated to be used in the given washing process in advance of the given iteration (block 312). In some cases, the amount of the wash fluid that is used in more than one subsequent iteration of the given washing process can be reduced.

In some cases, the one or more actions are performed only upon the temperature values for one or more earlier sampling periods of the sampling periods of the given iteration preceding the respective sampling period of the given iteration being greater than or equal to the predefined temperature and/or the predefined temperature range. Additionally, or alternatively, in some cases, the one or more actions are performed not only based on the temperature values for the respective sampling period of the given iteration being greater than or equal to the predefined temperature and/or the predefined temperature range but also on the values of readings of one or more additional sensors (e.g., conductivity sensors, optical sensors, flow sensors that indicate the rate of flow of the wash fluid) that are associated with respective milkmeters in the milkmeter system and that are obtained during the given iteration of the given washing process. In this regard, it is to be noted that the readings from one or more of these additional sensors can also be provided during a milking session. For example, a conductivity sensor associated with a respective milkmeter of the milkmeters in the milkmeter system can be used to provide conductivity level readings during a milking session, the conductivity level readings being indicative of a conductivity of milk that flows through the respective milkmeter during the milking session.

In some cases, the actions can include stopping the given iteration of the given washing process before the designated time for completion of the given iteration. In so doing, resources (e.g., wash fluid, electrical power (e.g., electricity for heating the wash fluid that is used in the given iteration), a time duration of the given iteration, etc.) can be conserved. In some cases, the given iteration can be immediately stopped upon the temperature values for the respective sampling period during the given iteration being greater than or equal to the predefined temperature and/or the predefined temperature range.

Additionally, or alternatively, in some cases, the actions can include designating a reduced amount of the wash fluid to be used in a subsequent iteration(s) of the given washing process in advance of the subsequent iteration(s), the reduced amount being less than the designated amount of the wash fluid designated to be used in the given washing process in advance of the given iteration of the given washing process. In so doing, resources (e.g., wash fluid, electrical power (electricity for heating the wash fluid that is used in the subsequent iteration(s)), a time duration of the subsequent iteration(s), etc.) can be conserved for the subsequent iteration(s).

In some cases, wash fluid conservation system 200 can be configured, e.g. using wash fluid conservation module 240, to calculate or determine an amount of the wash fluid that has flowed through the milkmeter system either: (a) during the given iteration of the given washing process or (b) until the end of the respective sampling period of the given iteration (the calculated or determined amount of the wash fluid is referred to herein as the “accumulated amount of the wash fluid”). In one example, the accumulated amount of wash fluid may be calculated using flow sensors in the milkmeter system that indicate the rate of flow of the wash fluid. The accumulated amount of the wash fluid can then be used to determine a reduced amount of wash fluid to be used in subsequent iteration(s) of the given washing process. In some cases, the reduced amount of the wash fluid to be used in the subsequent iteration(s) can be equal to the accumulated amount of the wash fluid. Alternatively, in some cases, the reduced amount of the wash fluid to be used in the subsequent iteration(s) can be: (a) greater than the accumulated amount of the wash fluid and (b) less than the amount of the wash fluid that (absent use of the information from the accumulated amount of wash fluid) would have been used in the subsequent iteration(s). Thus, through using the information contained in the accumulated amount of the wash fluid, as described herein, the system may, in one example, use less wash fluid than it otherwise would have.

In some cases, the accumulated amount of the wash fluid can be calculated or determined, at least in part, by calculating or determining a respective amount of the wash fluid that has flowed through each milkmeter of the milkmeters in the milkmeter system either: (a) during the given iteration of the given washing process or (b) until the end of the respective sampling period of the given iteration (e.g., using flow sensors that indicate the rate of flow of the wash fluid through the milkmeters).

It is to be noted that, with reference to FIG. 3, some of the blocks can be integrated into a consolidated block or can be broken down to a few blocks and/or other blocks may be added. It should be also noted that whilst the flow diagrams are described also with reference to the system elements that realizes them, this is by no means binding, and the blocks can be performed by elements other than those described herein.

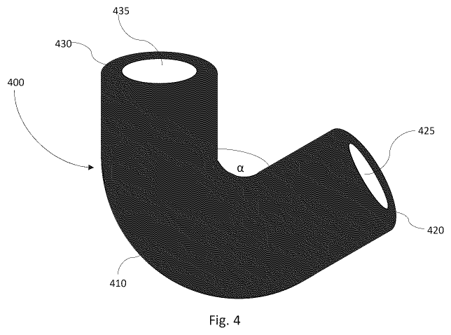

Attention is now drawn to FIG. 4, a schematic illustration of one example of a feed inlet 400 configured to be coupled to a milk channel 100 of a milkmeter, in accordance with the presently disclosed subject matter.

In accordance with the presently disclosed subject matter, feed inlet 400 can be configured to include a curved pipe 410 having a first end 420 and a second end 430. The first end 420 has a first opening 425, enabling a fluid that flows through a milk conducting hose (not shown in FIG. 4) to enter the curved pipe 410. In some cases, the fluid can be milk that is extracted from a non-human mammal during a milking session. In some cases, the fluid can be a wash fluid for cleaning the milking system, including, inter alia, the milk channel 100.

The second end 430 of the curved pipe 410 has a second opening 435. The second opening 435 enables the fluid that flows through the milk conducting hose and subsequently through the curved pipe 410 to exit the curved pipe 410 and enter the milk channel 100 of the milkmeter.

In some cases, the angle of curvature, a, between the first end 420 of the curved pipe 410 and the second end 430 of the curved pipe 410 can be less than about 90 degrees. In some cases, a can be between about 45 degrees to about 90 degrees. In some cases, a can be between about 50 degrees to about 75 degrees. In some cases, a can be between about 50 degrees to about 70 degrees (e.g., about 51, 52, 53, 54, 55, 56, 57, 58, 59, 60, 61, 62, 63, 64, 65, 66, 67, 68, 69, or 70 degrees).

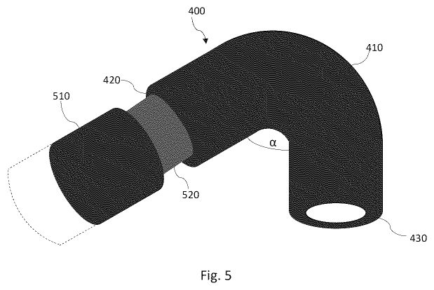

Attention is now drawn to FIG. 5, a schematic illustration of one example of a coupling of a feed inlet 400 to a milk conducting hose 510 and to a milk channel 100 of a milkmeter, in accordance with the presently disclosed subject matter.

In accordance with the presently disclosed subject matter, the feed inlet 400 is configured to be coupled to the milk conducting hose 510 and to the milk channel 100 (not shown in FIG. 5) to enable a fluid that flows through the milk conducting hose 510 to subsequently flow through the milk channel 100. In some cases, the feed inlet 400 can be coupled to a milk conducting hose 510 having a diameter of 16 mm. Alternatively, in some cases, the feed inlet 400 can be coupled, for example, to a milk conducting hose 510 having a diameter of 19 mm.

In some cases, as illustrated in FIG. 5, the feed inlet 400 can be configured to be coupled to the milk conducting hose 510 via a hollow adaptor 520 that extends between the milk conducting hose 510 and the feed inlet 400.

In some cases, the feed inlet 400 can be configured to be coupled to the milk channel 100 by connecting the second end 430 of the feed inlet 400 to an upper end of the milk channel 100 (not shown).

In some cases, the milk conducting hose 510 has a first cross sectional area and a section of the milk channel 100 (e.g., the section of the milk channel 100 illustrated in FIG. 1) has a second cross sectional area that is less than the first cross sectional area. It is advantageous to manufacture the section of the milk channel 100 to have a second cross sectional area that is less than the first cross sectional area of the milk conducting hose 510 since this allows for the milk channel 100 to be filled with a fluid (e.g., milk, a wash fluid, etc.) that is flowing through the milk channel 100 a greater percentage of the time during which the fluid is flowing through the milk channel 100, which can result in a more accurate calculation of an amount of the fluid that has passed through the milk channel 100. It is noted in this regard that the feed inlet 400 can be configured to connect to a milk channel 100 of a milkmeter, a milk sensor, or any other device in which there is a need or a benefit to having the milk channel 100 filled with a fluid (e.g., milk, a wash fluid, etc.).

In some cases, the angle of curvature, a, of the feed inlet 400 is selected to stream the fluid (e.g., milk, a wash fluid, etc.) to the milk channel 100 in bursts, i.e. the fluid is allowed to accumulate in the feed inlet 400 before it is streamed to the milk channel 100. By streaming the fluid to the milk channel 100 in bursts using the feed inlet 400 with the angle of curvature, a, the amount of time during the milking session and/or the cleaning procedure in which the milk channel 100 is filled with a fluid is increased. Moreover, by streaming the fluid to the milk channel 100 in bursts using the feed inlet 400 with the angle of curvature, a, the amount of the fluid that flows through the milk channel 100 during the milking session and/or the cleaning procedure can, at least in some cases, be more accurately calculated, at least in part due to the increase in the amount of time during the milking session and/or the cleaning procedure in which the milk channel 100 is filled with the fluid. In some cases, as detailed earlier herein, inter alia with reference to FIG. 4, a can be less than about 90 degrees. In some cases, a can be about 60 degrees.

It is to be understood that the presently disclosed subject matter is not limited in its application to the details set forth in the description contained herein or illustrated in the drawings. The presently disclosed subject matter is capable of other embodiments and of being practiced and carried out in various ways. Hence, it is to be understood that the phraseology and terminology employed herein are for the purpose of description and should not be regarded as limiting. As such, those skilled in the art will appreciate that the conception upon which this disclosure is based may readily be utilized as a basis for designing other structures, methods, and systems for carrying out the several purposes of the present presently disclosed subject matter.

It will also be understood that the system according to the presently disclosed subject matter can be implemented, at least partly, as a suitably programmed computer. Likewise, the presently disclosed subject matter contemplates a computer program being readable by a computer for executing the disclosed method. The presently disclosed subject matter further contemplates a machine-readable memory tangibly embodying a program of instructions executable by the machine for executing the disclosed method.

1. A wash fluid conservation system comprising a processing circuitry configured to:
for one or more given milkmeters of one or more milkmeters in a milkmeter system, obtain, for one or more sampling periods during a given iteration of a given washing process for cleaning the milkmeter system, temperature values that are indicative of a temperature of a wash fluid that is flowing through the respective given milkmeter; and
in response to the temperature values for a respective sampling period of the sampling periods being greater than or equal to a predefined temperature prior to a designated time for completion of the given iteration, perform one or more actions to reduce an amount of the wash fluid that is used in at least one of the given iteration or a subsequent iteration of the given washing process, subsequent to the given iteration, to be less than a designated amount of the wash fluid designated to be used in the given washing process in advance of the given iteration.

2. The wash fluid conservation system of claim 1, wherein the temperature values are obtained from one or more temperature sensors that are included in the given milkmeters.

3. The wash fluid conservation system of claim 1, wherein the actions include stopping the given iteration before the designated time.

4. The wash fluid conservation system of claim 3, wherein the given iteration is immediately stopped upon the temperature values for the respective sampling period being greater than or equal to the predefined temperature.

5. The wash fluid conservation system of claim 1, wherein the actions include designating a reduced amount of the wash fluid to be used in the subsequent iteration in advance of the subsequent iteration, the reduced amount being less than the designated amount.

6. The wash fluid conservation system of claim 5, wherein the processing circuitry is further configured to:
calculate or determine an accumulated amount of the wash fluid that has flowed through the milkmeter system either: (a) during the given iteration or (b) until the end of the respective sampling period;
wherein the reduced amount is designated to be greater than or equal to the accumulated amount.

7. A wash fluid conservation method, comprising:
for one or more given milkmeters of one or more milkmeters in a milkmeter system, obtaining, for one or more sampling periods during a given iteration of a given washing process for cleaning the milkmeter system, temperature values that are indicative of a temperature of a wash fluid that is flowing through the respective given milkmeter; and
in response to the temperature values for a respective sampling period of the sampling periods being greater than or equal to a predefined temperature prior to a designated time for completion of the given iteration, performing one or more actions to reduce an amount of the wash fluid that is used in at least one of the given iteration or a subsequent iteration of the given washing process, subsequent to the given iteration, to be less than a designated amount of the wash fluid designated to be used in the given washing process in advance of the given iteration.

8. The wash fluid conservation method of claim 7, wherein the temperature values are obtained from one or more temperature sensors that are included in the given milkmeters.

9. The wash fluid conservation method of claim 7, wherein the actions include stopping the given iteration before the designated time.

10. The wash fluid conservation method of claim 9, wherein the given iteration is immediately stopped upon the temperature values for the respective sampling period being greater than or equal to the predefined temperature.

11. The wash fluid conservation method of claim 7, wherein the actions include designating a reduced amount of the wash fluid to be used in the subsequent iteration in advance of the subsequent iteration, the reduced amount being less than the designated amount.

12. The wash fluid conservation method of claim 11, further comprising:
calculating or determining an accumulated amount of the wash fluid that has flowed through the milkmeter system either: (a) during the given iteration or (b) until the end of the respective sampling period;
wherein the reduced amount is designated to be greater than or equal to the accumulated amount.
